(19) United States
(12) Patent Application Publication
(10) Pub. No.: US 2020/0029901 A1
(43) Pub. Date: Jan. 30, 2020

(54) CONTACTLESS MEASUREMENT, MONITORING AND COMMUNICATION OF BODILY FUNCTIONS OF INFANTS AND OTHER HELPLESS INDIVIDUALS

(71) Applicant: Sameh Sarhan, Santa Clara, CA (US)

(72) Inventor: Sameh Sarhan, Santa Clara, CA (US)

(73) Assignee: XTRAVA INC, Santa Clara, CA (US)

(21) Appl. No.: 16/573,958

(22) Filed: Sep. 17, 2019

Related U.S. Application Data
(63) Continuation-in-part of application No. 15/453,911, filed on Mar. 9, 2017, now Pat. No. 10,470,669.

(51) Int. Cl. A61B 5/00; A61B 5/08; A61B 5/11; A61F 13/42

(52) U.S. Cl. CPC A61B 5/6808; A61B 5/0008; A61B 5/0816; A61B 5/11; A61F 2013/424; A61F 13/42; A61B 2562/0219; A61B 2562/0271; A61B 5/6804

(57) ABSTRACT

A health monitoring system contained within an intrinsically safe miniature wearable enclosure, for contactless measurement and communication of a variety of bodily functions of infants or other helpless individuals, utilizing novel sensors and data interpretation methods.

### CROSS-REFERENCE TO RELATED APPLICATION

Priority is claimed under 37 CFR 1.78 and 35 USC 119(e) to U.S. Provisional Application 62/306,463 (XT1603101), filed 10 Mar. 2016).

### CONTINUATION-IN PART APPLICATION

This application repeats a substantial portion of prior application Ser. No. 15/453.911, filed Mar. 9, 2017, having received a Notice of Allowance Jun. 18, 2019, and adds and claims additional disclosure not presented in the prior application. Since this application names the inventor named in the parent application, it may constitute a continuation-in-part of the that application.

### TECHNICAL FIELD

This disclosure relates generally to wearable electronic measurement, monitoring, and communication equipment. More specifically, this disclosure relates to wearable electronic measurement, monitoring and automated communication equipment for infants, babies, children, and adults who may not be able to adequately care for themselves.

### BACKGROUND

During the past several years, there have been many products introduced to allow monitoring of infants and others for reasons of safety. Parents and others are able to monitor subjects who are located in different rooms of a dwelling or in completely different locations. Wired intercom systems that existed more than 60 years ago have been replaced by or supplemented with handy wireless equipment. More recently, communication for monitoring purposes has been supplemented with remote monitoring of bodily functions such as breathing and heart rate. Remote vital sign monitoring of cardiac and other patients by professional organizations has been supplemented with low cost equipment for consumers.

### SUMMARY

This Summary is provided as a general introduction to the disclosure providing the detailed description and figures. It summarizes some aspects of the disclosed invention. It is not a detailed overview of the disclosure and should not be interpreted as necessarily identifying key elements of the invention, or otherwise characterizing the scope of the invention disclosed in this Patent Document.

This disclosure covers novel ideas to expand upon existing equipment types that measure and remotely monitor bodily functions and environment. Additional ideas, when implemented, permit the monitoring equipment and systems to be more practical. For instance, the apparatus (“monitor”) and overall system disclosed herein, to measure and communicate life signs and other functions of infants and others “subjects”, is not attached directly to or in contact with the subject. Moreover, it is not necessarily attached to any diaper, but rather, to any layer of the subject's clothing. Novel and/or modern scientific and engineering methods, used singly and in combination, make this contactless improvement possible.

The portable, wearable, and non-intrusive/non-invasive health monitoring system described in the instant disclosure is contained in an attractive badge-sized, light-weight, and safe enclosure that easily attaches to the outside of inner clothing, i.e. makes no contact with the skin. All sensors, measurement circuitry, power source and management, signal processing, data storage, and wireless communication circuitry are contained within the enclosure. Multiple quantities and types of sensors “distributed sensing” provide redundancy and reliability of bodily function measurements and facilitate removing the need for direct bodily contact.

Bodily functions that could be measured, monitored, and communicated, including in real time, include temperature, heart rate, electrocardiogram, blood glucose concentration, breathing rate, coughing, sneezing, wheezing, gagging, edema, obstructed airway, reflux, regurgitation, vomiting, miscellaneous motion, position, water retention, bladder abundance, clothing wetness, stomach abundance, and sleep patterns. Measurements of environment may include sounds, temperature, and light, including ultra violet. Apparatus to implement these measurements and communication to the user could include multiple 3-axis accelerometers, magnetic eddy current sensors, electrostatic sensing elements, microphones, temperature sensors, light/UV sensors, and RF Wireless. Interpretation of raw data could be implemented with the aid of advanced automated machine learning techniques. Machine learning and other artificial intelligence and calculation tasks could be distributed among the apparatus attached to the subject's clothing and other system building blocks, such as local video and communication devices and even the user's smart phone.

Other aspects, features and advantages of the invention will be apparent to those skilled in the art from the following Disclosure.

### BRIEF DESCRIPTION OF DRAWINGS

For a more complete understanding of this disclosure and its features, reference is now made to the following description, taken in conjunction with the accompanying drawings, in which:

FIG. 1 shows various subjects wearing example health monitors attached to clothing at various example body locations.

FIG. 2 shows examples of information about said subjects that could be measured by health monitors shown in FIG. 1 and wirelessly communicated locally and thence world-wide.

FIG. 3 shows details of an example enclosure and mechanism to safely attach said health monitor to any clothing layer.

FIG. 4 shows the side of said example health monitor that faces away from said subject, with the mounting rails closed but without being attached to clothing. Also shown are possible locations of various example sensors and RF communications antenna.

FIG. 5 shows the side of said health monitor that faces toward said subject, showing possible locations of various example sensors that gather information about him/her.

FIG. 6 shows another example health monitor 610, as also present in FIG. 1, 131; along with its special clip 620 used to attach said health monitor to clothing.

FIG. 7A through D show in sequence how said health monitor is attached to clothing.

FIG. 8 shows a block diagram of the health monitor attached to a Human Body, as well as other elements of an example overall system.

FIG. 9A, 9B displays prior work that established, by application of ECG probes, potential gradients on the skin surface.

FIG. 10 displays prior work showing that body surface potential variations can be used to derive rate and other features of respiration.

FIG. 11 shows measured respiration data from an accelerometer contained in a wearable monitor designed by the inventor.

FIG. 12 shows measured respiration data from a static electric field sensor contained in a wearable monitor designed by the inventor.

FIG. 13 shows measured respiration data from an electrometer sensor contained in a wearable monitor designed by the inventor.

FIG. 14 demonstrates actual humidity and possible diaper wetness level data from a live subject, using an electrostatic field sensor.

FIG. 15 demonstrates actual diaper wetness level data from a live subject, using the same electrostatic field sensor and assisted by accelerometer data and behavioral analysis to report out the actual condition.

### DETAILED DESCRIPTION

The various figures, discussed below, and the various embodiments used to describe the principles of the present invention in this patent document are by way of illustration only and should not be construed in any way to limit the scope of the invention. Those skilled in the art will understand that the principles of the invention may be implemented in any type of suitably arranged device or system. For instance, although several figures show the Instrument enclosure with a shape that could be construed to resemble a butterfly or other winged creature, the enclosure can have a wide variety of shapes. The attachment mechanism shown and later described is but one example of a safe means to attach the Instrument to clothing.

In general, this disclosure describes apparatus (“health monitor”) that attaches to the clothing of human and animal subjects (“subject”), obtains real time information pertaining to a significant variety of bodily functions and environmental conditions, and communicates said information immediately or later to interested parties and/or recording equipment (“user” or “caregiver”) who may be co-located or at any remote location.

In general, this disclosure provides the description of a novel, multiply complementary sensor set, data management and communication blocks contained in a non-intrusive, non-invasive portable and wearable health monitoring system enclosure, to detect and characterize respiration, clothing wetness, bodily motion, temperature and sleep patterns.

Depending on the implementation, the techniques implemented in said health monitor can provide significant benefits in a range of fields, such as the care of infants, babies, children and adults, and detection and monitoring of unfavorable health conditions indicated by specific bodily motions, incontinence, respiration rates, patterns, volume, and depth.

BREATHING MONITORING AND ANALYSIS FUNCTION: Apparatus to monitor the breathing of subjects has existed for many years. “Breathing is one of the most obvious signs of human vitality and activity; however, it can also reflect the status of a patient and the progression of an illness. The entire process, from inhalation to the exhalation, is referred to as the breathing or respiration cycle (“RC”). Respiratory rate indicates the number of breaths per unit time, such as one minute. Any alterations in the respiratory rate can help predict potentially serious clinical events, such as a cardiac arrest, or it may suggest that a patient be admitted to an intensive-care unit.” (Cretikos, M. A.; Bellomo, R.; Hillman, K.; Chen, J.; Finfer, S.; Flabouris, A. Respiratory rate: The neglected vital sign. Med. J. Aust. 2008, 188, 657-659)

It is considered important to monitor the time pattern, volume, and depth of respiration of a variety of subjects, such as infants, athletes, truck drivers, soldiers in the field, as well as those already known to suffer from dangerous medical conditions. It has also been demonstrated that detailed knowledge of breathing characteristics supports the ability to diagnose disease conditions. Such knowledge can be used to recommend therapeutic procedures to prevent or forestall further permanent medical damage and/or death. Moreover, as respiration monitoring products become more convenient and economical, it will be used routinely, including by those who are ill but are initially unaware of it.

Historically, respiratory measurements have not only lacked the cost requirements and convenience to encourage every-day and every-night out-of-clinic use, but they have been unavailable to infants and children, who do not tolerate invasive or intrusive equipment. People in general do not choose intrusive and/or inconvenient monitoring systems for themselves or loved ones, unless there is already a strong indication of adverse medical conditions.

Examples of intrusive and/or invasive respiration measurement systems include belts with strain sensors, nasal air flow rate and/or temperature sensing during the breathing cycle, and processed electrocardiogram data. Examples of non-intrusive respiration measurement systems include photographic methods and ultra-wideband pulse radar. These latter systems are neither portable nor wearable. Clearly there is a need for a respiration measurement instrument system that is wearable, non-invasive, non-intrusive, and portable.

CLOTHING WETNESS DETERMINATION: In all human cultures where infants, babies, and toddlers wear clothing, the issue of determining when underclothing such as diapers need to be changed represents a continuous nuisance, medical risk, expense, and waste of time. A similar issue exists for infirm adults, especially those who cannot communicate.

Often, babies are aroused from sleep unnecessarily to check a diaper, causing harm to them and disruption to caregivers. During automotive travel, it may be necessary to pull over and stop the vehicle in what may be unsafe or inclement conditions, just to determine whether a diaper needs to be changed. If a diaper is checked too late, the consequences could be painful and dangerous chafing and/or infiltration to other clothing and surrounding objects. The diaper wetness monitoring task is magnified when there are multiple babies or infirm patients.

Many sensing methods that automatically provide an alarm when a diaper has become wet have been advanced during the past twenty years or so. All of them require special diaper construction and/or devices that mount inside the diaper or other undergarment and make physical contact with excrement.

In contrast, the methods described in the instant disclosure require no special clothing construction and employ only miniature electronic devices that are either hand-held or attached to the outside of a diaper or other undergarment. The single device can be used repeatedly for a period of years with no maintenance, and it could include communication with the caregiver via wireless means. Moreover, the device itself and method of attachment to clothing are intrinsically safe for infants and babies.

The present example embodiments include three main sensors and a temperature sensor, having complementary uses. They detect clothing wetness, detect and characterize breathing, depending upon the position and activity of the wearer, as well as the relative motion of the wearer and clothing. The detected and processed signals are of sufficient quality to monitor several critical aspects of respiration: breathing rate, depth, volume, and pattern. These sensors draw extremely low power and are intrinsically safe. They induce no electric currents within the body and make use of naturally occurring physical effects within the skin. In addition to said principal function, these sensors could be used to detect and communicate, whether or not said monitoring system is or is not attached to a subject.

One sensor is a three-axis accelerometer that operates successfully during periods when the clothing to which the monitoring system enclosure is attached is very thin and tight against the wearer/subject's body. A second sensor measures the electric fields between its electrodes and the body. It is useful when the monitor is up to several centimeters from the body, such as when attached to the outside of a diaper. The third sensor operates efficiently when the clothing is separated from the skin within the range of 1 to 10 centimeters, such as on the outside of loose, hanging clothing. Its set of electrodes develops electric potential differences that are caused by naturally occurring potential differences on the skin, through the intermediary of the electric field created by the latter.

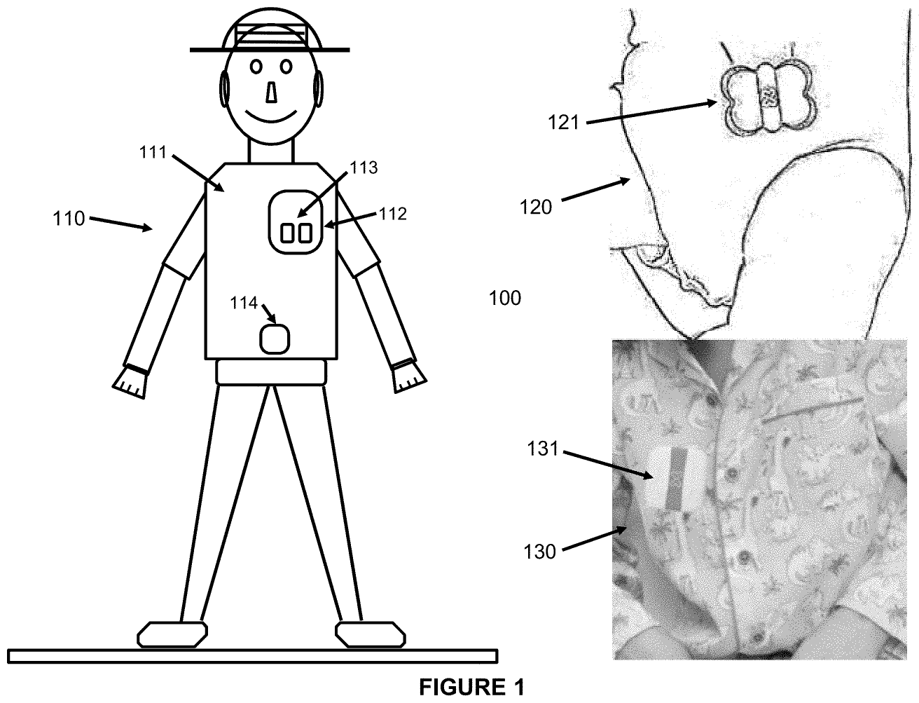

FIG. 1 illustrates a basic wearable multiple sensor bodily function monitor assembly on three subjects 100. This monitor assembly could be attached to clothing worn by a baby, a child, an adult, or even an animal. The subject, 110 is wearing an undergarment 111, with a health monitor assembly 112 that contains a multiplicity of sensors used for breathing and other bodily functions, such as wetness of clothing. One said sensor, 113, is designed to register the existence of an electric field having some component in the horizontal direction by creating an electric potential difference between the illustrated electrodes. Sensor 113 and the remaining sensors (not shown) are connected to measurement and processing circuitry as well as wireless communications circuitry. Monitor assembly 112 is smaller, relative to the size of subject 110, than the one shown in the figure. Not shown in the figure are any over-garments that cover the undergarment 111 and Monitor 112, allowing the latter to be comfortable and unobtrusive. Monitor assembly 114 is shown attached to clothing at another example bodily location. Subject 110 is depicted as robust and in a standing position but could be as an example infirm and asleep.

Subject 120 is an infant. Although his monitor assembly looks like a decoration or toy (in this example resembling a butterfly or other small winged creature) and is as safe as a properly designed toy, it is loaded with technology that communicates with the outside world and assesses a host of bodily functions and environmental conditions. Safety features include but are not limited to enclosure materials used in toys, the lack of sharp corners, a size that is too large for a baby to swallow, and an ultra-safe level of electromagnetic emissions. Subject 130, a small child, is wearing a similar monitor assembly, but the enclosure is not designed to resemble a toy.

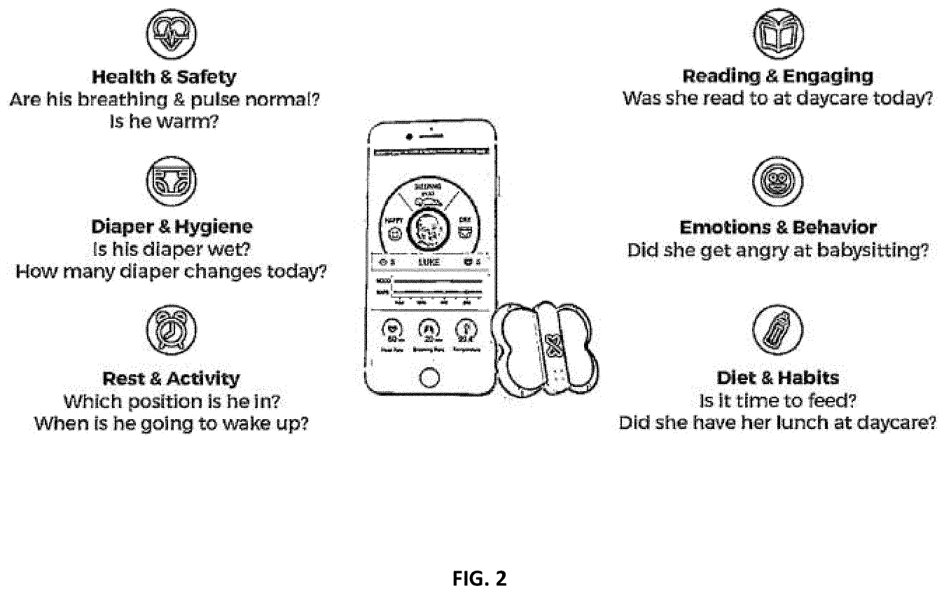

FIG. 2 illustrates an example health monitor assembly user interface employing a smart phone, and the monitor assembly itself. It also shows a partial list of service functions performed with the aid of said assembly.

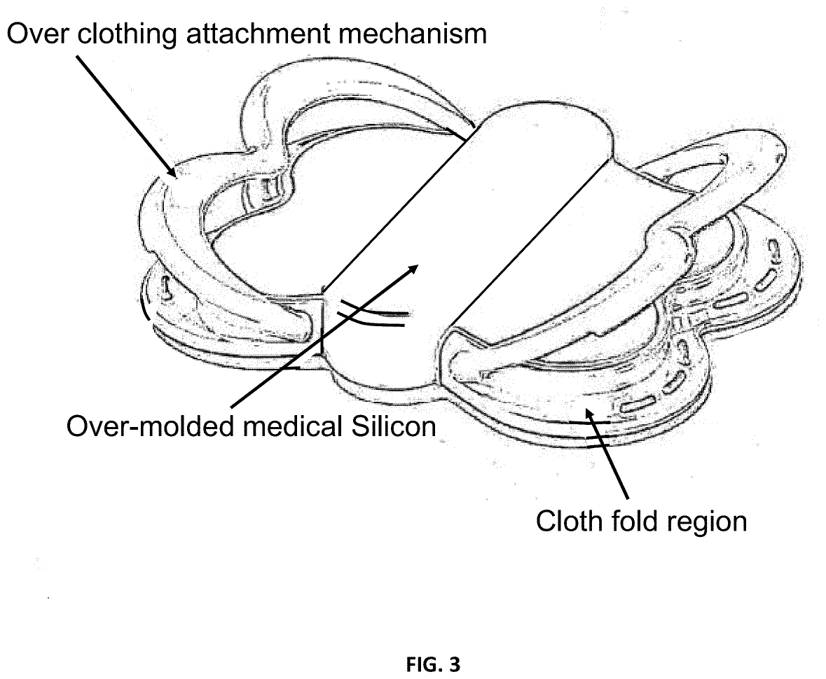

FIG. 3 shows an example health monitor apparatus enclosure with means for attachment to clothing. The monitor is shown as seen from the side that faces away from the Subject. The overall size is large enough that it cannot be swallowed by a baby or small child. The material composition is a “medical” Silicon that is used for teething toys. The clothing cloth fits into the fold region at the edges of the “wings”, and the attachment mechanisms, shown in the open state, clamps to the clothing.

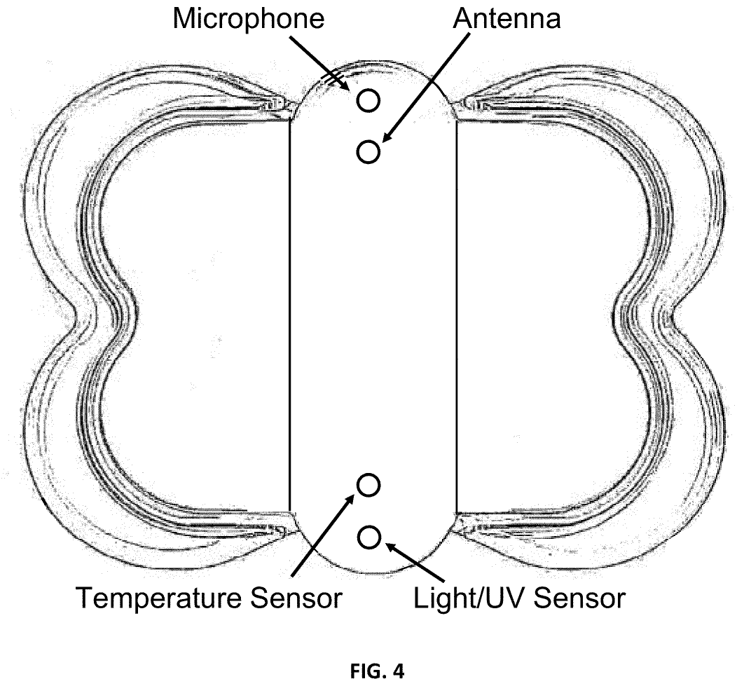

FIG. 4 also shows the health monitor, containing sensors, measuring circuitry and data handling circuitry as seen from the side that faces away from the Subject. The attachment mechanism is shown in the closed condition. Also shown is the region where the RF communications antenna and various example facilities may be located inside the enclosure. The Light/UV sensor is located at one end of the Instrument, as an indicator of normal ambient light on the Subject, as well as damaging ultra-violet radiation. An additional sensor for infrared radiation could also be added. At the other end of the health monitor is the microphone that could monitor ambient sounds of the Subject and others. Next to the light/UV sensor is an ambient temperature measurement sensor. For the light, temperature, and sound sensors, the Silicon enclosure has modified properties to pass light and sound and temperature information but still maintain integrity such as waterproofing and safety.

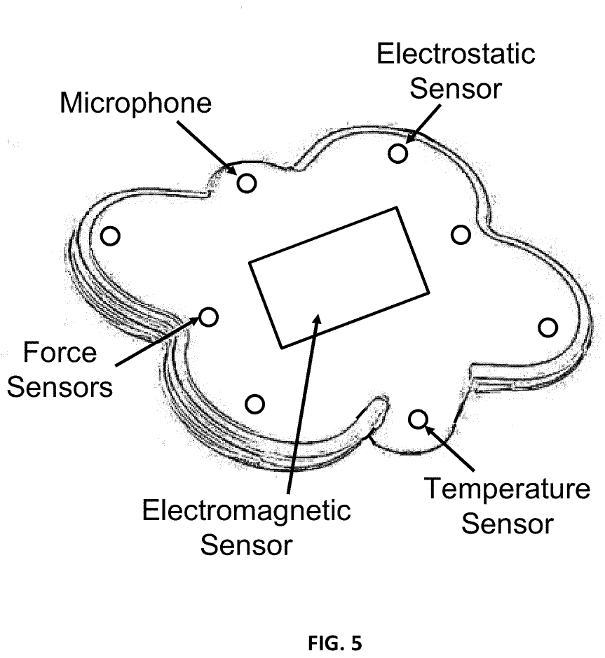

FIG. 5 shows the side of the health monitor that faces the subject. The microphone on this side picks up internal sounds from the subject's body, and the temperature sensor picks up internal “core” body temperature. The electromagnetic and electrostatic sensors within the enclosure could pick up motion and properties of internal organs and fluids. The items marked “force sensors” are 3-axis accelerometers that also help determine motion of internal organs, but mainly sense motion and position of the body as a whole.

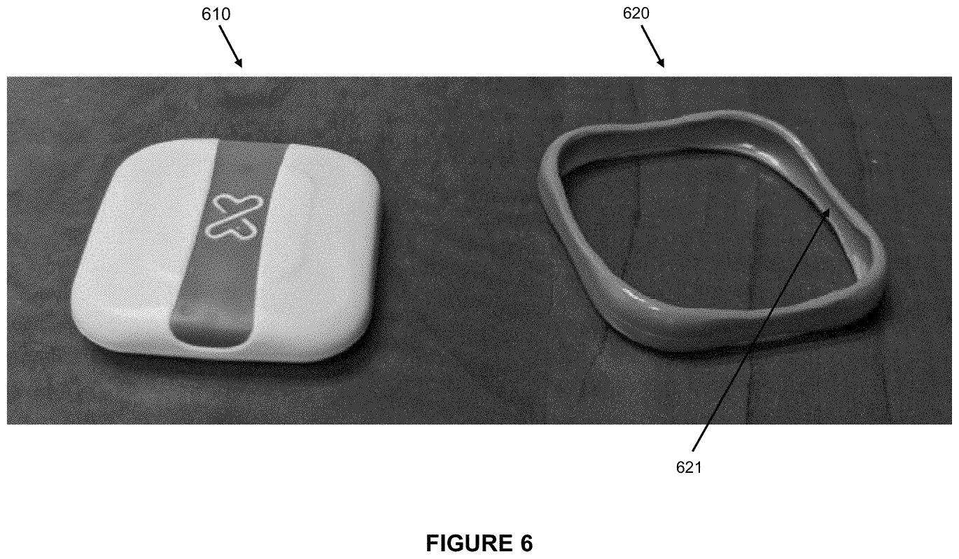

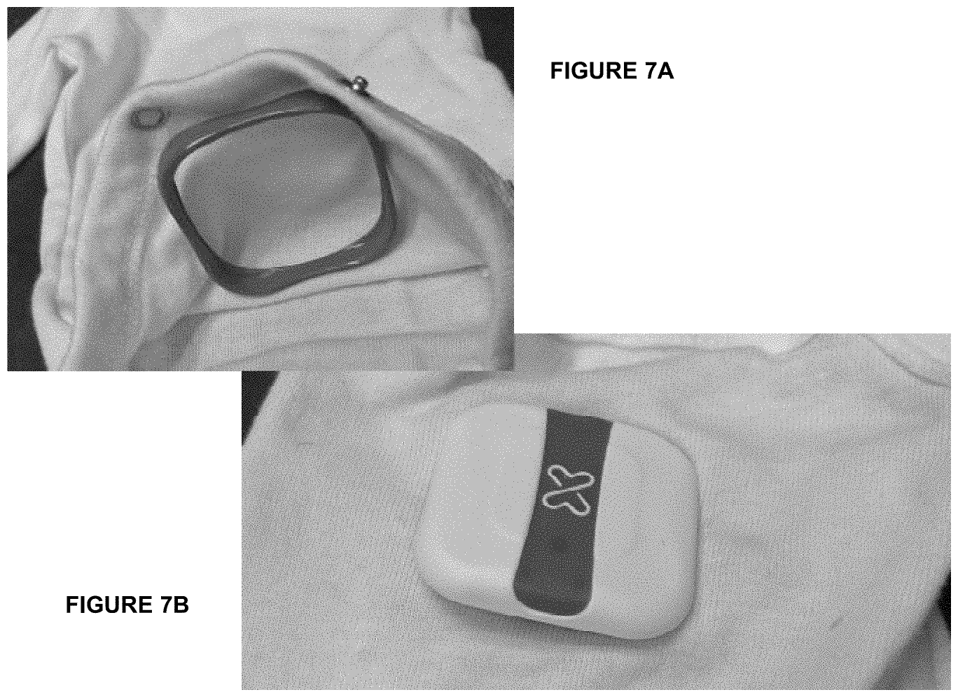

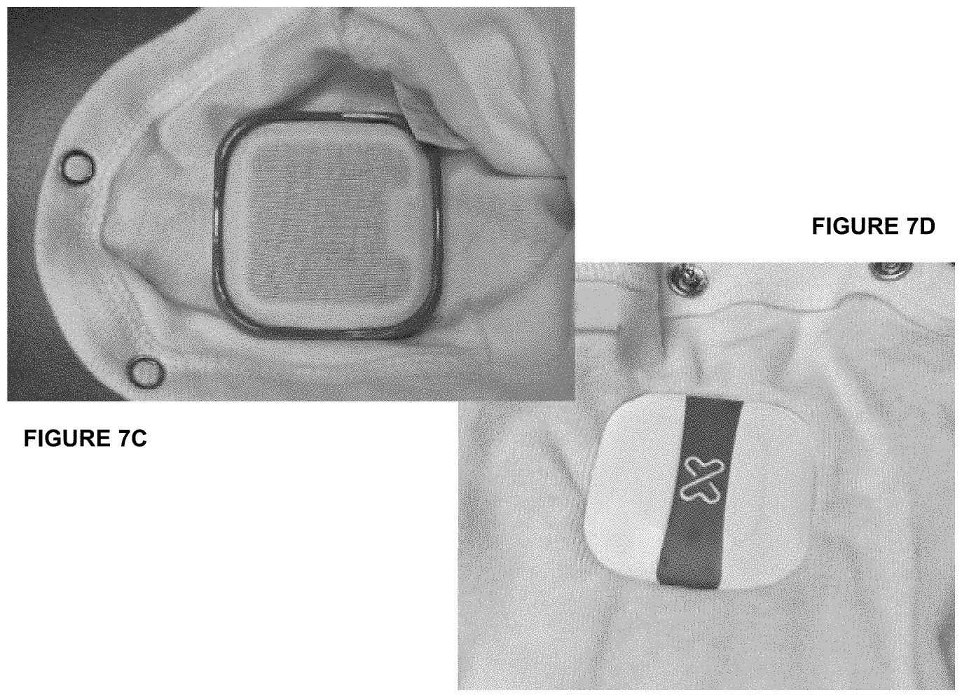

One enclosure example—previously shown being worn by the small child, 130, in FIG. 1—is explained in FIGS. 6 and 7. FIG. 6 shows closeups of health monitor top view 610, and its clip 620. The health monitor is attached to a clothing layer using this clip that is easy to apply and remove. However, all corners of the clip are keyed 621 in such a way that a baby cannot remove it. The clip attaches to the monitor from the opposite surface of the clothing layer, capturing the clothing. Normally, the monitor is attached to the outside of a clothing layer. The clip can be in any orientation with respect to the monitor, rotated or flipped. The monitor operates from any angle and orientation and is completely self-contained.

FIG. 7A through 7D shows the sequence of steps used to attach the monitor to clothing. FIG. 7A shows the clip in position under the clothing layer to accept the monitor. FIG. 7B shows the monitor placed on the outside of the clothing layer, directly over the clip, ready for attachment. FIG. 7C shows the underside of the clothing with the clip attached to the monitor, visible through the trapped clothing layer. FIG. 7D shows the attached monitor from the outside of the clothing layer.

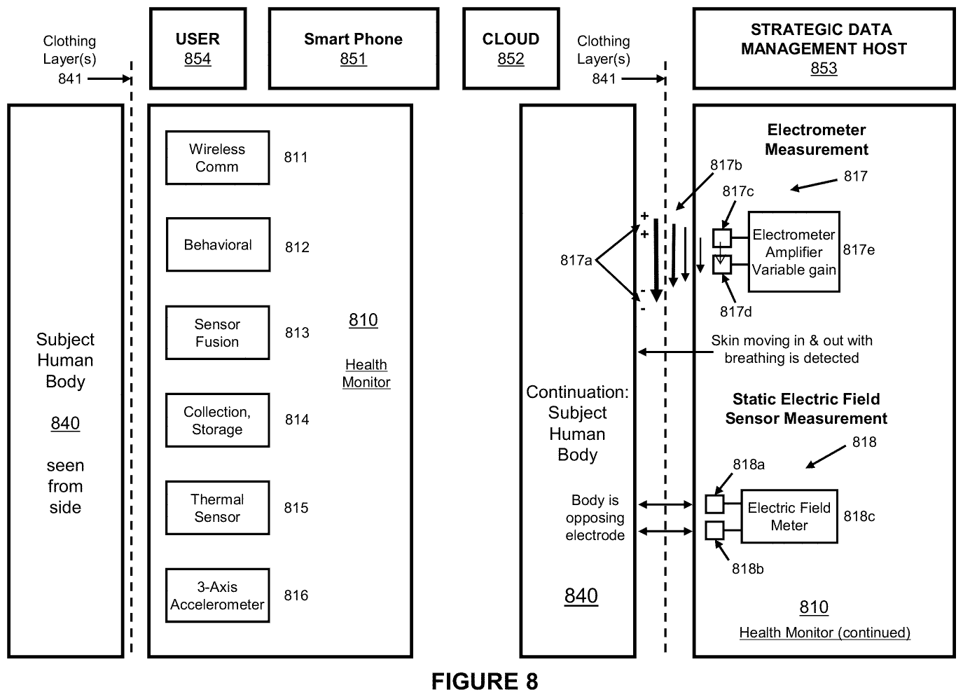

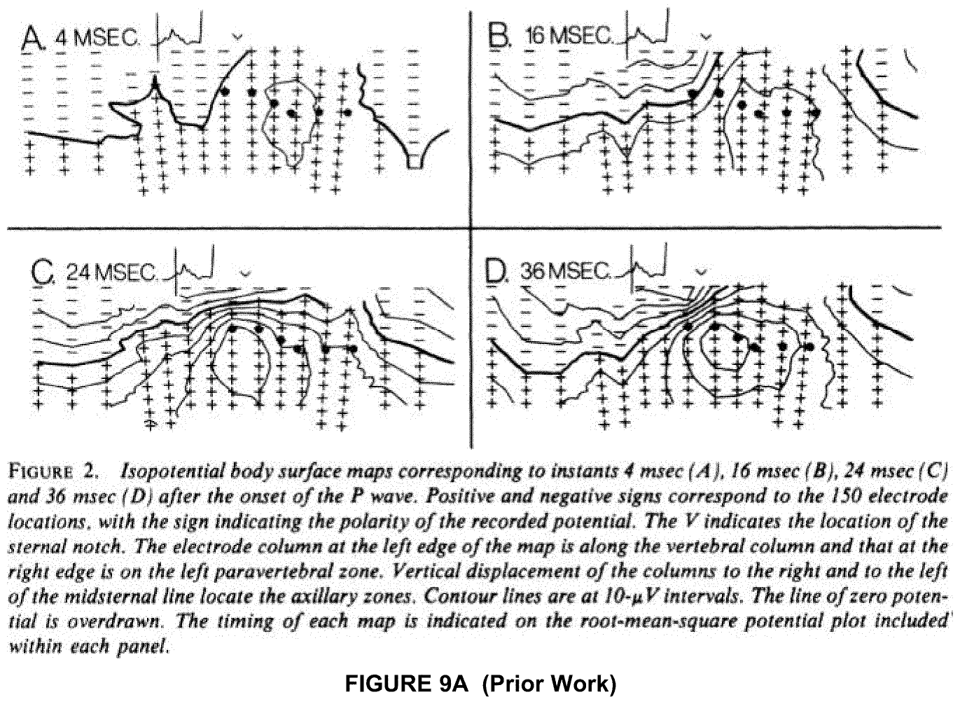

FIG. 8 shows a block diagram of a subject, i.e. human body 840 wearing health monitor 810 on the outside of one or more clothing layers 841, plus the entire system that permits short and long term practicality. Health monitor 810 is fully self-contained and functions independently, collecting and storing 814 data from sensors 818, 817, 816, and 815. It also processes and interprets said data using sensor fusion 813 and behavioral 812 layers. Omitted from the figure is the rechargeable battery and power management block contained within said health monitor. At such times that smart phone 851 or other suitable RF communication device capable of short range, low power service is within range and otherwise available, monitor 810 sends the data it has collected, stored, and analyzed and receives any pertinent information. Smart phone 851 stores the data and presents updated interpreted results in the form of text, graphics and alarms to user 854, who could be a caregiver. Smart phone 851 or other suitable device could also communicate through “cloud” 852 with strategic data management host 853. Such communication could provide interpretative data service and means to download updated firmware to health monitor 810. Smart phone 851 or other suitable device could communicate with said health monitor and use cloud 852 to further communicate with a user/caregiver (not shown) at a remote location. All data transferred to and from smart phone 851 to cloud 852 is encrypted to provide privacy for medical and other data.

The four types of said sensors work separately and together and with said data processing including behavioral analysis to provide information about the subject's respiration, temperature, wetness of clothing, and sleep patterns. Thermal sensor 815 can determine subject's changes of orientation during sleep, where sleeping on top of said sensor causes higher temperatures and rapid temperature increase. It may also assist in detection of excrement emergence. The 3-axis accelerometer sensor 816 detects breathing when health monitor 810 is in close contact with the body surface, including on the outside of thin clothing. It is also used for detecting motion patterns such as turning over in sleep, falling, having a diaper changed, and demonstrating to behavioral processor 812 that diaper was not changed during condition for which the electric sensors 817 and 818 cannot function.

If health monitor 810 is separated from the body surface but not by more than about 5 centimeters, the static electric field measurement block 818 detects breathing and the wetness of clothing. Electrodes 818a and 818b of sensor 818, attached to electric field meter 818c each work in conjunction with the body surface to measure static electric field that is modulated by sensor-to-body distance and also humidity. The temporal pattern of electric field changes indicates breathing characteristics and wetness of clothing. Accelerometer 816 data is processed to provide motion sensor indications of whether or not monitor 810 is in a suitable location relative to the body to obtain reliable humidity data from electrostatic sensor 818. Once the electric field has been reduced owing to the high humidity of wet clothing, motion data from accelerometer 816 allows behavioral layer 812 to ascertain that the clothing is still wet, even though electrostatic sensor 818 later indicates dry air when the subject's position relative to loose clothing is unfavorable for the static electric field sensor's operation. The advanced processing circuitry in blocks 812 and 813 distinguishes the breathing curve and wetness condition from any miscellaneous motion of loose clothing. This static electric field sensor measurement block 820 operates in both low and high humidity environments.

As the ambient humidity can have a very wide range of values, behavioral layer 812 constantly monitors static electric field level to determine the fiduciary level; so the variations from respiration and clothing wetness are each recorded in differential fashion.

When the subject is active and wearing loose clothing, separation of the subject's body surface and health monitor may exceed several centimeters. In this case, the electrometer measurement block 817 which operates with a separation of up to about 10 centimeters, is utilized. It has been shown that electrical potential gradients 817a exist on the skin, which are correlated with aspects of the breathing cycle, such as depth. Potential gradients 817a on the skin surface cause electric fields 817b that extend beyond the skin surface. If the there is a finite component of the electric field 817b vector in the direction along the axis of the electrode assembly 817c, 817d, a potential (voltage) exists between these electrodes. Said voltage is detected with extremely high impedance (electrometer) amplifier 817e that includes an automatic gain control feature to handle a large range of signal levels.

It should also be mentioned that the extremely high impedance sensors used in said health monitor are not only effective but provide a high measure of safety for the subject; as only the minutest amount of currents can exist within the body. Moreover, these sensors use almost no current to operate, thus maximizing battery life.

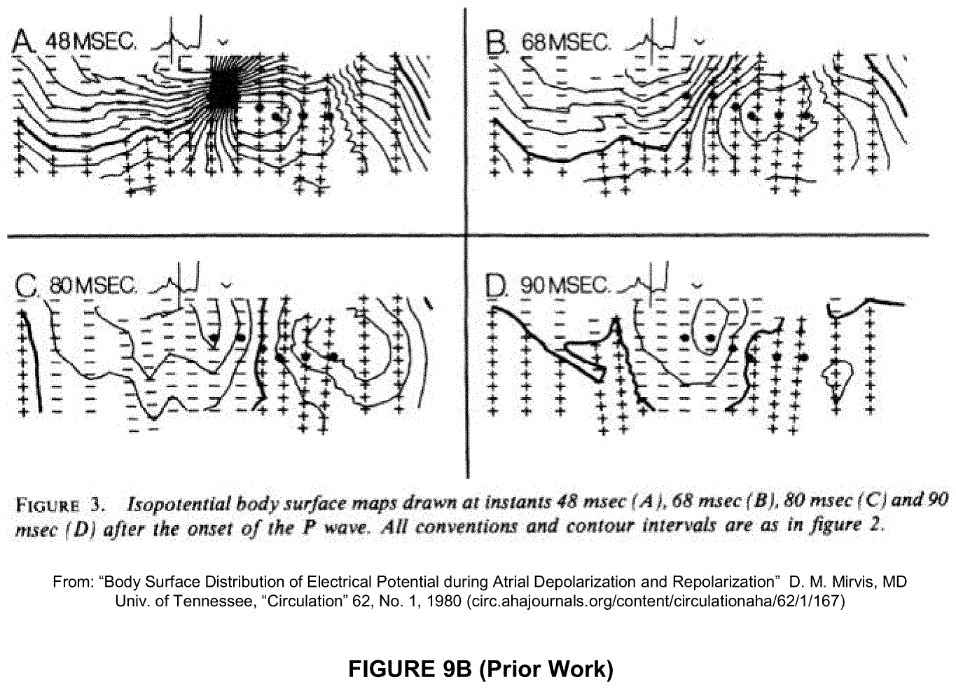

FIG. 9A, 9B displays prior work that established by application of ECG probes the existence, magnitudes, and spatial and time patterns of said potential gradients on the skin surface. The captions therein adequately describe the measurement results and are not repeated in the instant Specification text.

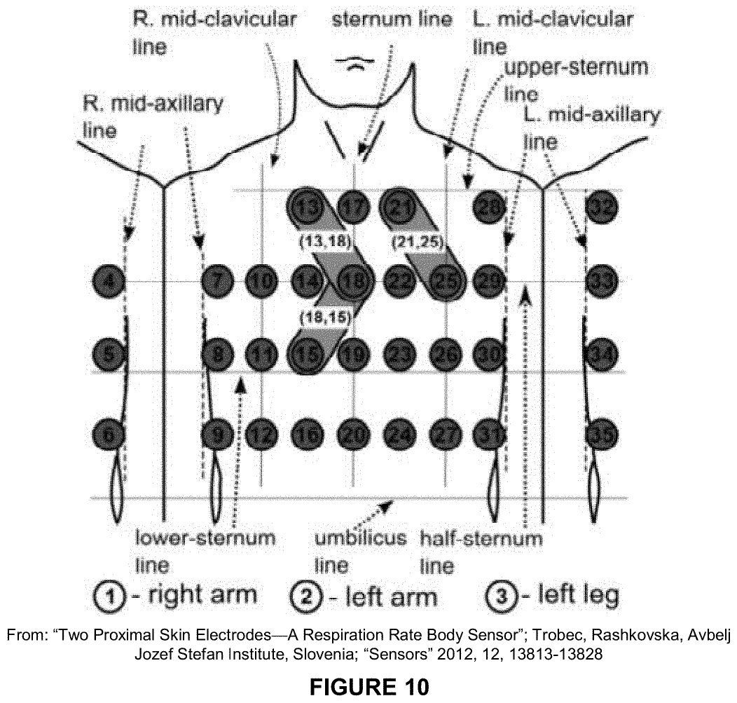

FIG. 10 displays prior work showing that body surface potential variations, measured by a pair of ECG-type electrodes, can be used to derive rate and other features of respiration. A 37-lead ECG system was used to identify the most favorable proximal location pairs to probe.

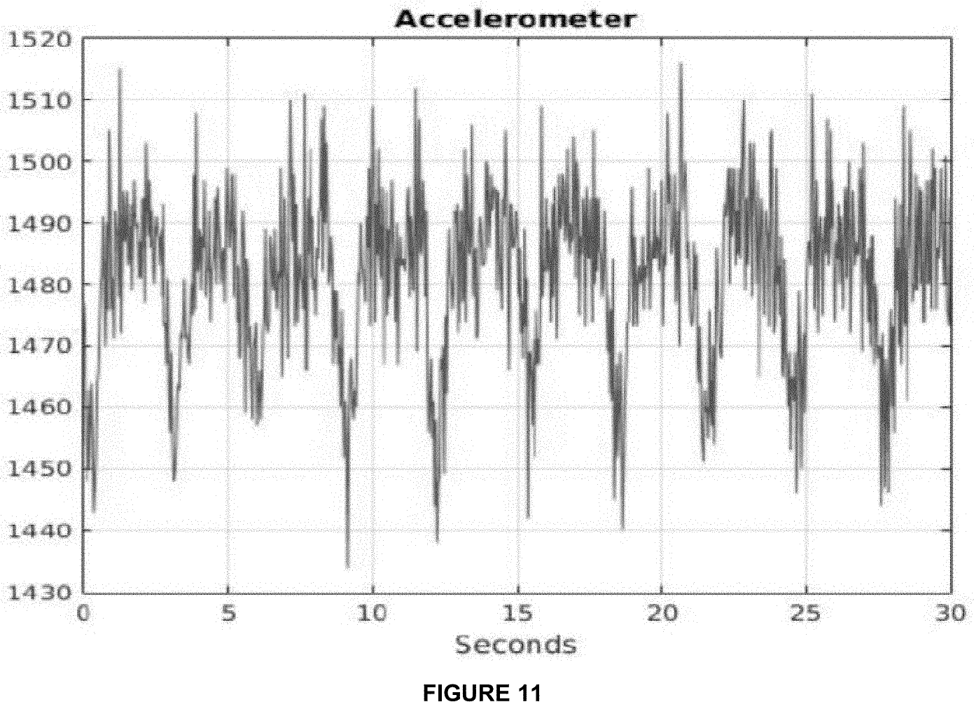

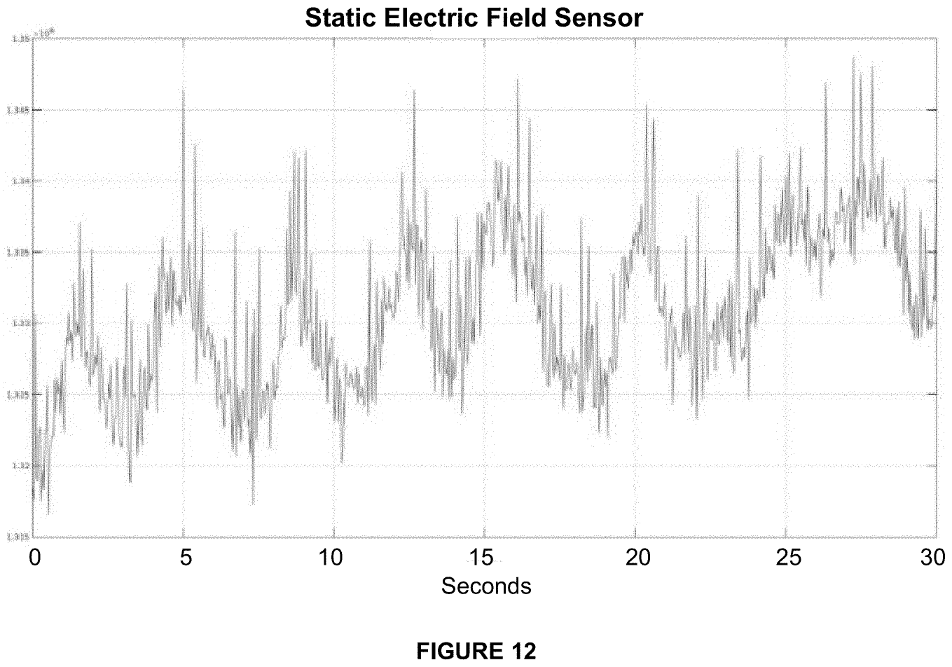

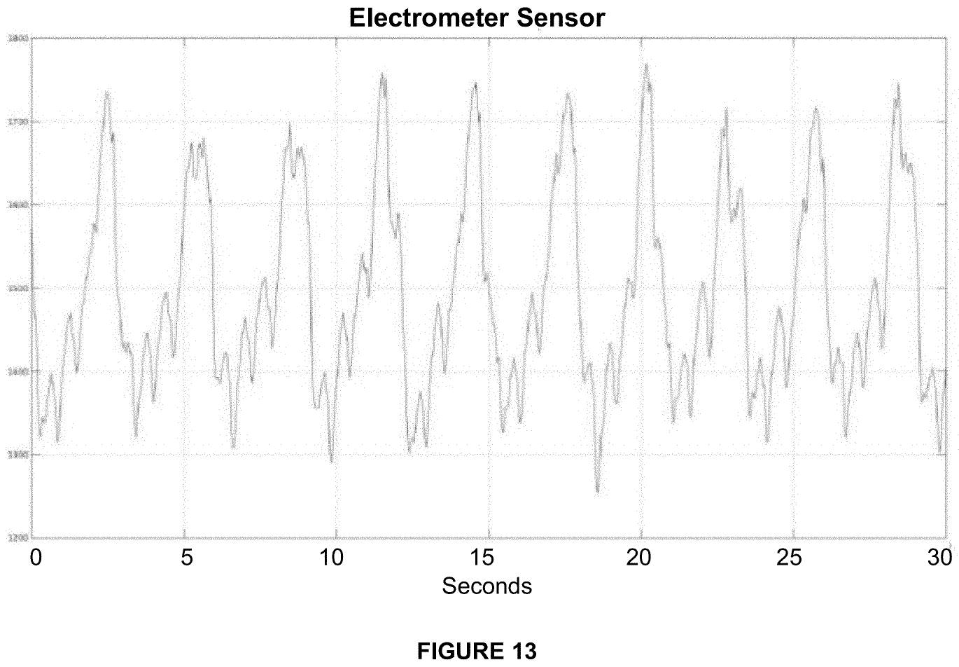

FIGS. 11, 12, and 13 show measured respiration data from the accelerometer measurement module 810, static electric field measurement module 820, and electrometer measurement module 830 respectively. Said data was acquired from said measurement modules as part of the complete portable, wearable and non-intrusive/non-invasive health monitor system 800 as described in the instant disclosure. The data is raw, prior to the extensive cleanup processing contained with said monitoring system. The linear vertical scales shown are arbitrary. Hidden in the data may be pulse rate and/or ECG information, which may be recoverable using various additional processing methods.

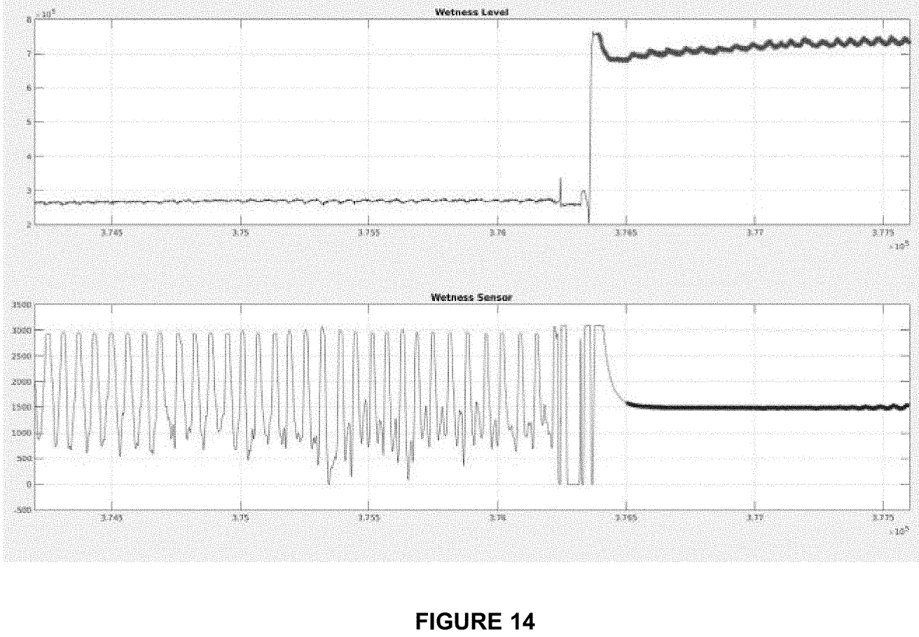

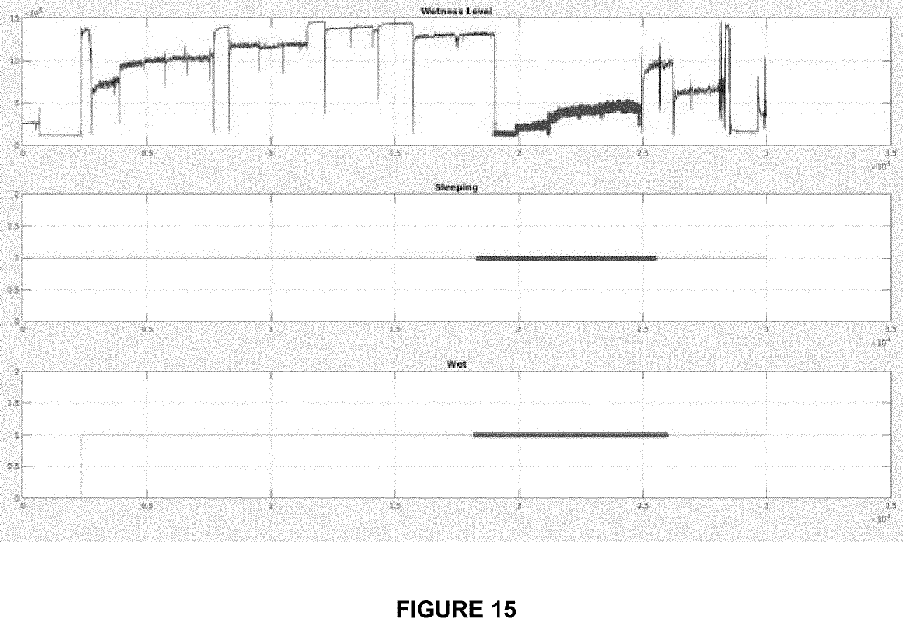

FIGS. 14 and 15 show wetness level and sleep data from a health monitor undergoing beta testing with a real subject. As explained for FIG. 8, the electrostatic sensor measures distance variations over a breathing cycle as the body expands and contracts, reducing the distance between the subject's skin and the sensor during inhale and increasing the distance during exhale. The sensor instrument feeds a fixed amount of current that goes from one electrode to the other. The current circulates from the current source to one electrode to and through the skin, to the other electrode, and back to the current source. The electric field created between the electrodes and the skin surface is perpendicular to the skin surface. At the end of the time period, the voltage between the electrodes is measured. Within a certain range of distances, the voltage level is a direct function of the distance between the electrodes and the skin surface.

For the clothing wetness level measured by humidity, for a given distance between the skin and electrodes, the higher the voltage at the end of the fixed current and time period, the lower is the humidity. When the clothing becomes wet, the humidity rises, and the voltage level between the electrodes at the end of the current injection time period becomes lower. As long as the humidity is low (dry diaper), the voltage between the electrodes continues to increase until the current fed between them ceases, whereupon the voltage drops with the current. When the diaper becomes wet, the humid air polarizes in such a direction as to create its own electric field in the opposite direction as that created by the current source; so the voltage cannot rise to its previous value in the standard amount of current injection time.

The top trace in FIG. 14 shows the result of digital processing that follows said basic electrostatic sensor measurements. As stated earlier in the instant disclosure, ambient humidity has a wide range of values. Moreover, as the sensor-to-subject distance varies with clothing movement, this sensor reading changes. Clearly, an absolute measurement scale from this sensor is not useful; therefore, a method that compares the average reading during an appropriate period prior to a wetness event is used. The top trace of FIG. 14 shows the output of a digital integrator. At the time this sensor shows a significant dry-to-wet indication, there is virtually no cause but that of actual wetness.

The bottom trace of FIG. 14 shows simultaneously for the same live test the output of the electrometer sensor (FIG. 8, block 818). As previously explained for FIGS. 8 through 11, regions of potential differences exist on the skin surface, creating an electric field parallel to the body that extends up to about 10 cm away. Electrometer 818 captures the value of said electric field. When the humidity rises, these potential differences are short circuited by the more conductive skin.

FIG. 15 illustrates use of the health monitor's behavioral block. The top trace shows the electrostatic sensor output. It shows a dry diaper at the beginning and then wet until almost 2.0 time-units on the horizontal scale. From that time until 2.5 time-units it shows “dry”, and then wet again. The accelerometer sensor signal (not shown) indicates no change of diaper during the entire period. (The middle trace just shows “sleeping” as a digital command output.) What really happened from 1.9 to 2.5 time-units is that the baby moved to a position to disable the electrostatic sensor outputting to the top trace. The bottom trace is a digital command that shows a Low at the beginning for “dry”, and a digital High level thereafter for “wet”. For the duration of this record, the subject baby wet its diaper at 0.25 time-units, and it was never changed.

The details provided in the instant specification describe particular implementations of systems for portable, wearable and non-intrusive/non-invasive respiration monitoring. Other embodiments could be implemented in any other suitable manner.

The different types of sensors, and multiple sets of sensors of the same type provide measurements of many bodily functions as well as provide additional accuracy, redundancy and reliability of said measurements. They also provide the ability to eliminate or reduce the effects of artifacts, such as those caused by the relative motion of the health monitor mounted on loose clothing and subject's body.

The sensors and communication devices located within this Instrument apparatus emit fields that are intrinsically safe as defined by recognized safety standards. Emissions from the Instrument that permeate the Subject's body are several orders of magnitude smaller than the minimum recommended by the Federal Communications Commission and other widely recognized standards and regulatory institutions and agencies.

Other embodiments could be implemented in any other suitable manner. For example, other enclosure materials than that mentioned in this disclosure may have all the desired properties to be safe for babies. The health monitor apparatus may have different sizes, shapes, and fastening mechanisms, and may be placed in different positions on the body. The clothing could be a “onesie” piece as shown or any other type or style.

The details provided in the foregoing description and figures describe in part particular implementations of the systems for performing the functions explained in this disclosure. Other embodiments could be implemented in any other suitable manner. For example, the figures show a particular module physical size and other physical configurations. This disclosure and as-built example equipment report on and utilize a standard interface package—the size, as opposed to other sizes and shapes. These configurations are for illustration only. Other embodiments could use different key system blocks, depending upon the implementation. Moreover, measuring pulse and respiration rate within the body only two examples of what the methods of this disclosure can perform. Other embodiments could be implemented in any other suitable manner. For example, this disclosure describes particular sizes, shapes, and other values. These values are for illustration only.

It may be advantageous to set forth definitions of certain words and phrases used throughout this patent document. The term “couple” and its derivatives refer to any direct or indirect communication between two or more elements, whether or not those elements are in physical contact with one another. The terms “transmit,” “receive,” and “communicate,” as well as derivatives thereof, encompass both direct and indirect communication. The terms “include” and “comprise,” as well as derivatives thereof, mean inclusion without limitation. The term “or” is inclusive, meaning and/or. The phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, have a relationship to or with, or the like.

While this disclosure has described certain embodiments and generally associated methods, alterations and permutations of these embodiments and methods will be apparent to those skilled in the art. Accordingly, the above description of example embodiments does not define or constrain this disclosure. Other changes, substitutions, and alterations are also possible without departing from the spirit and scope of this disclosure, as defined by the following claims.

What is claimed is:

1. A health monitoring system that permits non-invasive measurement and wireless reporting of bodily functions of a subject wearing a diaper or other underwear and an outer layer or layers of clothing outside the diaper or said underwear, the system comprising:
a plurality of sensors, a processor, and a memory, the memory containing machine readable instructions configured to be executed by the processor to measure the bodily functions using the plurality of sensors;
a square, thin enclosure for housing all components of the system, including the sensors, the processor, the memory, a power management circuitry and the wireless circuitry, the enclosure having an associated coupling clip being configured to sandwich any layer of clothing to removably couple the enclosure to the clothing layer without necessitating any modification to the clothing layer;
where the associated coupling clip can be used in any orientation with respect to the enclosure, is easy to install, but cannot be removed by a baby;
wherein, every measurement of the bodily functions is configured to occur without any direct contact between a skin of the subject and the enclosure.

2. A system as defined in claim 1, where the enclosure includes Silicon.

3. A system as defined in claim 2, where data from multiple sensors therein is processed and used together by a behavioral firmware layer to accurately assess status of the subject.

4. A system as defined in claim 3, where all functions and data storage are contained within the system until a wireless storage means is available.

5. A system as defined in claim 4, where the sensors include
an accelerometer to assess motion of the subject;
an electrostatic field module that quantifies respiration features and clothing wetness by inducing and measuring an electric field perpendicular to the subject's skin surface;
an electrometer module that quantifies respiration features, clothing wetness, and relative motion of clothing and the subject, by measuring an electric field parallel to the subject's skin surface;
a thermal sensor that assists with determination of the subject's clothing wetness and body orientation.
